SEQUENCE LISTING (26 sequences)

SEQ ID NO: 1 - DNA, 1234 bp, Salmonella typhimurium
  CDS (1)..(717)
atgattaaagtgctgattgtggatgatgagccgttagcgcgggaaaatctgcggattttgctccaggggcaggatgacattgagattgtgggagagtgcgcgaacgcggtagaagcgattggcgcggtacataagttgcgacctgatgtgctgtttctggatattcagatgccgcgtatcagtggactggagatggtaggaatgcttgatccggaacaccgcccgtatatcgtttttttaaccgcgtttgacgaatacgccatcaaagcctttgaagaacacgcttttgattatctgctcaagccgatagaggagaaacggctggaaaaaacgttacatcgtctgcgtcaggagcgcagtaaacaggatgtttcgttgttgccggaaaaccagcaggcgcttaaattcattccctgtaccggacacagccggatctatttgttgcaaatggatgatgtcgcctttgtcagtagccgtatgagcggcgtttatgtgaccagcagtgaagggaaagaggggtttaccgagctgacgctgcgcacgctggaaagccggacgccgctactgcgttgtcatcgtcagtttctggtgaatatggcccatttgcaggaaatacggctggaggataatgggcaggcagagctgattttacgcaacggcctgacggtgccggtaagccgtcgctatctgaaaagtttaaaagaggcgattggcctgtaaaagactgttagaatatcgttttgccatagaaacgaccgaaggcctcatgctgagtaacgatattctgcgtagcgtgcgctacattttaaaagctaataataccgatctggcgcgtatcctggcgctgggtaacgttgatgctacgccggagcagattgcaatctggttgcgcaaagaagaggaagaggggtttcagcgttgcccggatatcgtgttgtcctcatttctcaatggcctcatttatgaaaaacgcggcaaagatgaggcggcgcctgcattgacggcggaacgtcgtatcaacaacaatattgtgctgaaaaagctgcgtatcgccttttcgctaaaaacagatgatatcctggcgatacttaccggtcagttgtttcgtgtctcaatgccagagatcaccgcgatgatgcgcgcgccggaccataagaacttccgcgaatgcggcgatcagtttatgcgttattttctgcgcggtctggcagcccgtgaacacgcggcgaagtaa

SEQ ID NO: 2 - protein, 239 aa, Salmonella typhimurium
MIKVLIVDDEPLARENLRILLQGQDDIEIVGECANAVEAIGAVHKLRPDVLFLDIQMPRISGLEMVGMLDPEHRPYIVFLTAFDEYAIKAFEEHAFDYLLKPIEEKRLEKTLHRLRQERSKQDVSLLPENQQALKFIPCTGHSRIYLLQMDDVAFVSSRMSGVYVTSSEGKEGFTELTLRTLESRTPLLRCHRQFLVNMAHLQEIRLEDNGQAELILRNGLTVPVSRRYLKSLKEAIGL

SEQ ID NO: 3 - protein, 155 aa, Salmonella typhimurium
MLSNDILRSVRYILKANNTDLARILALGNVDATPEQIAIWLRKEEEEGFQRCPDIVLSSFLNGLIYEKRGKDEAAPALTAERRINNNIVLKKLRIAFSLKTDDILAILTGQLFRVSMPEITAMMRAPDHKNFRECGDQFMRYFLRGLAAREHAAK

SEQ ID NO: 4 - DNA, 933 bp, Salmonella typhimurium
  CDS (1)..(933)
atgattgccgccatactcagtacgaccacaatggccaatgccagtagccttgctatttcagtggcgaatgatgatgcaggtatatttcaaccatccttaaatgccctttatgggcacccggctgcagaccggggtgattatactgccgggctctttttaggctacagccacgatctcactgacgccagtcaactctcttttcatatcgcgcaagatatttattctccatcaggcgccaataagagaaagcctgaagccgtcaaaggcgaccgcgcgttcagcgcctttcttcataccggactggagtggaattcattagccaccaattggttacgttatcgtttaggtaccgatattggcgttattggtcctgacgcaggcggtcaggaggttcagaatcgggcgcatcgaataataggcgcagaaaaataccctgcctggcaggatcaaattgaaaaccgttatggttacacggcaaaaggaatggtgtcccttacgccagctattgatattctgggcgtcaatgtgggcttctatccagaagtatctgctgtcggcggcaatctcttccagtatttggggtacggcgcaaccgttgcgctgggaaatgataagaccttcaactctgataatgggtttgggttactgtcccgccgagggttaatacacactcaaaaagagggattgatctataaagtatttgctggcgttgagcgcagggaagttgataaaaactacacgttacaggggaaaacgctgcaaacaaaaatggaaacggtggatattaataaaaccgtggatgaatatcgcgtcggtgccaccatcggctattctcccgtcgcattttctctttctttaaacaaagtcacgtcggaatttcgcaccggagacgactattcttatatcaacggagacatcaccttcttcttctga

SEQ ID NO: 5 - protein, 310 aa, Salmonella typhimurium
MIAAILSTTTMANASSLAISVANDDAGIFQPSLNALYGHPAADRGDYTAGLFLGYSHDLTDASQLSFHIAQDIYSPSGANKRKPEAVKGDRAFSAFLHTGLEWNSLATNWLRYRLGTDIGVIGPDAGGQEVQNRAHRIIGAEKYPAWQDQIENRYGYTAKGMVSLTPAIDILGVNVGFYPEVSAVGGNLFQYLGYGATVALGNDKTFNSDNGFGLLSRRGLIHTQKEGLIYKVFAGVERREVDKNYTLQGKTLQTKMETVDINKTVDEYRVGATIGYSPVAFSLSLNKVTSEFRTGDDYSYINGDITFFF

SEQ ID NO: 6 - DNA, 450 bp, Salmonella typhimurium
  CDS (1)..(450)
atgtcatcaggtaatatgctggctatcttttatttcctattggaagggattggtaatacgctgcttgtaacatttacctgttttctctcagcgtttttgaccgggcttacagtcgccgtattacggcgactatcccctcttccactacagaagatactcgacgtactggtctttattcttcggggaattccaatacttattgctgtttttcttgtttattttggattgccatcgattggcatatatgtttcaccactggtggccatgaacctcagcgtcggcttaatcagcgggagttatcttgccgaggtgttcagaggggcgcttaaattagttgagccattcgaaataacggtagcgaaagtggcaggaatgcgtcaattacaggttataataaatattcgaactgccacagatgttgcgattttctgtgcacggtattatcaatga

SEQ ID NO: 7 - protein, 149 aa, Salmonella typhimurium
MSSGNMLAIFYFLLEGIGNTLLVTFTCFLSAFLTGLTVAVLRRLSPLPLQKILDVLVFILRGIPILIAVFLVYFGLPSIGIYVSPLVAMNLSVGLISGSYLAEVFRGALKLVEPFEITVAKVAGMRQLQVIINIRTATDVAIFCARYYQ

SEQ ID NO: 8 - DNA, 957 bp, Salmonella typhimurium
  CDS (1)..(201)
atgtataacaatgaacctggcgctcagtccgatccaacgttgggctacacttttcaaaatgattttctggcattaagccaggcgttttcactacccgaaatagattataccgatatttcccaacgcgaacagttggcggcggcgattaaaagatggccgctcctggctgaatttgcgcaaccacattcattaaggaagccatgaatggcgattctgggactgcagggcgtgcgcggcggggtgggaaccacatctctcaccgcagcactcgcctgggcgttacaaatcttaggggaaaatgtcctggtgattgacgccagccccgataacctgctgcgcatgtcgtttaacgtcgacttcgtccatcagggcggttgggcgcgatcgttgctggacggccaggactggcgcgatgccggattacgttatacctcacagcttgatctactgccgtttggacagctcaccgcgcaagagcgggaaaatccgcagtcctggcaggagacgctgggagagatcggatcggctatccaggcgctgaaagcgagcgggcgttatagctggattttactggacttgccctacggcgcgtcaccgctgacacggcaactggtgagcctctgcgaccatacgctggcgatagcccgggttgacgctaactgccatattcgccttcatcagcaggcgttgcctgccggagcgcatattctgatcaatgacttacgcattggcagtcagttacaggacgatctgtaccaggtctggctgcaaagccagcgccggttgctgcccattgtgatccatcgcgatgaggcgatggcggagtgcatggcgtcgaaacagccgttaggcgaatatcgcagcgattcgttagccgccgaagaggtgttaacgctggcgaactggtgcctgctgcatgatgcgggcgacaaaacgtccgccgggagcctgcgatga

SEQ ID NO: 9 - protein, 67 aa, Salmonella typhimurium
MYNNEPGAQSDPTLGYTFQNDFLALSQAFSLPEIDYTDISQREQLAAAIKRWPLLAEFAQPHSLRKP

SEQ ID NO: 10 - protein, 250 aa, Salmonella typhimurium
MAILGLQGVRGGVGTTSLTAALAWALQILGENVLVIDASPDNLLRMSFNVDFVHQGGWARSLLDGQDWRDAGLRYTSQLDLLPFGQLTAQERENPQSWQETLGEIGSAIQALKASGRYSWILLDLPYGASPLTRQLVSLCDHTLAIARVDANCHIRLHQQALPAGAHILINDLRIGSQLQDDLYQVWLQSQRRLLPIVIHRDEAMAECMASKQPLGEYRSDSLAAEEVLTLANWCLLHDAGDKTSAGSLR

SEQ ID NO: 11 - DNA, 1836 bp, Salmonella typhimurium
  CDS (1)..(1836)
atggagagtgttatgaaatttaaatatgctttaacttctctcgcattatctgttgcaattttgtcatcagtaccttctactgctttcgccatcggcggcgccagcggcgctaaagtggactatcaggtccagggaaaaattggcgaagttgttatgaacccctatgatatcgcgccgctaaccgccgttattcgtaatggcggttaccagttacgtgacgtgcatgtacggattgtacccaaagaaaatggccaggagatcgcgtataaagttaataataaataccttttaacgtatggcggtattcccgtctttggtctttacccggattatgtcaataccgttgaagttgaatatacaagtatccagggtagtaaaaccgaaaatgtaaaagaaagctataaaatgtatgcaccgcctgcctatattgaatcagcgggtacaaaagaagaacaatcagcactctttactatcgatgttaaaaaggtttccccagaatttaaagatcgcttgtatcttttgaataatacgaaagataagtctgggaatggaacgcgtactgtctggaacaaccctactgggggtgcattagaatggaacttcactacagctaacgctattatcgacacctccggtgatattcgttggtttatgaatccaagttcaatttatgatttaaagtcaatttatcgtgctggcgttatgatgggctttaaacaaaacaaggatggcgcactatcgtggggctacggtcagcgttatgtgaaatacgatatcatggggcgtgaaatctttaaccgccgcctgccggataattataacgatttttcacactcaatggataacgcggccaacggtcactacttcctgcgtgtagccagctctaactataaacgccctgatgggaaaaatgttcgtaccgtgcgtgatgtgattgccgaagttgatcagaacggcgtggtagtggatgaatggcgtctgtttgatatcctcgatccttatcgtgatgtgataatgaaaaccctcgatcagggcgctgtgtgcctgaatatcgacgccagccagtccggccatacgttgagcgaagaagatctggcggcgctggactcctccgacaaattcggggatatcgtgggtagtggggctggccgcaactgggcgcatgtcaacagcgtcgactatgacagtgaagatgattccatcatcatcagctcccgccaccagagtgcgattatcaaaatcggccgcgataagaaagtgaagtggatactgggtacgcctgctggctggaaagcgccatttaatgccgcaattctgacgcctgtggatagcaaaggccaaaaaatcgcctgccaggacagtggctgcgagggtgacttcgactggacatggacgcaacatacggcctttaaaattgatagtaagagtaaaggcgatatcttatacctttccgctttcgacaatggcgatggccgcggcttagaacagcctgctatgcagagtatgaaatacagccgctccgtgatttacaaaatcgaccagaaaaacaagacagtccaacagatctggcaatacggtaaagagcgcgggaacgagtggtttagcccggtaacctctatcaccgagtaccagactgacaagaattctgtgttcgtgtattccgcaacagcaggtggtgcgtttgatttatcggtaggcgcatttaccagcttgcctaatccgtatctggaagagttcaaatggggagaaaaagagcctgcggttgaaatgcaaatacatggtgcgcgtggatatcaggctatgccatttagcctgaccaaagcgcttactgagtag

SEQ ID NO: 12 - protein, 611 aa, Salmonella typhimurium
MESVMKFKYALTSLALSVAILSSVPSTAFAIGGASGAKVDYQVQGKIGEVVMNPYDIAPLTAVIRNGGYQLRDVHVRIVPKENGQEIAYKVNNKYLLTYGGIPVFGLYPDYVNTVEVEYTSIQGSKTENVKESYKMYAPPAYIESAGTKEEQSALFTIDVKKVSPEFKDRLYLLNNTKDKSGNGTRTVWNNPTGGALEWNFTTANAIIDTSGDIRWFMNPSSIYDLKSIYRAGVMMGFKQNKDGALSWGYGQRYVKYDIMGREIFNRRLPDNYNDFSHSMDNAANGHYFLRVASSNYKRPDGKNVRTVRDVIAEVDQNGVVVDEWRLFDILDPYRDVIMKTLDQGAVCLNIDASQSGHTLSEEDLAALDSSDKFGDIVGSGAGRNWAHVNSVDYDSEDDSIIISSRHQSAIIKIGRDKKVKWILGTPAGWKAPFNAAILTPVDSKGQKIACQDSGCEGDFDWTWTQHTAFKIDSKSKGDILYLSAFDNGDGRGLEQPAMQSMKYSRSVIYKIDQKNKTVQQIWQYGKERGNEWFSPVTSITEYQTDKNSVFVYSATAGGAFDLSVGAFTSLPNPYLEEFKWGEKEPAVEMQIHGARGYQAMPFSLTKALTE

SEQ ID NO: 13 - DNA, 894 bp, Salmonella typhimurium
  CDS (1)..(894)
atggcaaaagcattgcaaggcgtaatggcggcgttgttgaccccatttgatcatcagcagcaactggatagcgaaagcctgcgccgtctggtgcgttttaacatcgggcagggtatcgacgggctgtacgttggcggttccaccggggaagcattcgtacagagccttgctgaaagagagcaggtactggagattgtcgccgaagaggcgaagggaaaaatcacgttgatcgcccatgtcgggacagtaagcaccgcagaaagccagcagcttgccagcgccgcaaagcgttacggttttgatgcggtctctgcggtgacgcctttttattaccctttcagttttgaagagcactgtgaccattatcgggcaatcattgattccgcagatggattgccgatggtggtatacaacattccggcgttaagcggtgtaaaacttaccctcgatcagatcaatacgctggtgacattaccgggcgtgagcgcgctgaagcaaacctccggcgatctcttccagatggagcagatccgccgcgcgcatccggatctggtgctgtacaacggttatgacgaaatcttcgcttccggcctgctggcgggcgcggacggcggtatcggcagcacatacaacattatgggctggcgttaccaggggattgtgcaggcattacgtgagggcgatgtggcgaaagcgcagcgtctgcaaaccgagtgcaataaggttattgatttactgattaaaaccggcgtattccgcggcctgaaaacggtcctgcattatatggacgtggtatcggtgccgctgtgtcgtaaaccgttcgcgcctgttgatgaaaaatacctgccggcgctcaaggcgctggctcaacaactgatggaagaaaaggcgtaa

SEQ ID NO: 14 - protein, 297 aa, Salmonella typhimurium
MAKALQGVMAALLTPFDHQQQLDSESLRRLVRFNIGQGIDGLYVGGSTGEAFVQSLAEREQVLEIVAEEAKGKITLIAHVGTVSTAESQQLASAAKRYGFDAVSAVTPFYYPFSFEEHCDHYRAIIDSADGLPMVVYNIPALSGVKLTLDQINTLVTLPGVSALKQTSGDLFQMEQIRRAHPDLVLYNGYDEIFASGLLAGADGGIGSTYNIMGWRYQGIVQALREGDVAKAQRLQTECNKVIDLLIKTGVFRGLKTVLHYMDVVSVPLCRKPFAPVDEKYLPALKALAQQLMEEKA

SEQ ID NO: 15 - DNA, 804 bp, Salmonella typhimurium
  CDS (1)..(804)
atgaacgcattgttatcaaatccttttaaagaaggattacgtaaaggggatacgcaaattggtttgtggctcagttcaaccacctcttatatggcggagattgcggcgacgtcgggttatgactggttgttaatagatggcgagcacgcgcctaatacggtgcaggacttgtatcatcagttacaggccatcgccccatacgccagccagccggtgattcgcccgatcgagggaagtaaagcgctcattaaacaggtactggacatcggggcgcaaacgctactgatcccgatggttgacaccgcagaacaggcgcgccaggtcgtttccgctacccgatacccgccgttggggcaacgaggcgttggcgcgagcgtggcgcgtgcggcgcgctgggggagaatcgacaactatatggcacaggccaatgagtcgctctgcctgttggtgcaggttgaaagcaaagtggcgctggaaaaccttgacgcgattctggaggttgaaggcattgatggcgtctttatcggtccggcagatctctctgcttcattgggttaccccgataatgccggacacccggaagtgcagcgaatcattgaagcgtgtatttatcgtattcgcgccgccggaaaagcggcgggttttttggcggtcgatccggcaatggcgcagaaatgtctggcgtggggcgcgaattttgtcgcggtcggggtagatacgatgctctacaccgaggcgctggacagcaggttggcgatgtttaaatctgttcagtcagtaagcacggcaaaacgtagttattga

SEQ ID NO: 16 - protein, 267 aa, Salmonella typhimurium
MNALLSNPFKEGLRKGDTQIGLWLSSTTSYMAEIAATSGYDWLLIDGEHAPNTVQDLYHQLQAIAPYASQPVIRPIEGSKALIKQVLDIGAQTLLIPMVDTAEQARQVVSATRYPPLGQRGVGASVARAARWGRIDNYMAQANESLCLLVQVESKVALENLDAILEVEGIDGVFIGPADLSASLGYPDNAGHPEVQRIIEACIYRIRAAGKAAGFLAVDPAMAQKCLAWGANFVAVGVDTMLYTEALDSRLAMFKSVQSVSTAKRSY

SEQ ID NO: 17 - DNA, 948 bp, Salmonella typhimurium
  CDS (1)..(948)
atggcttctttagttattgctgaacataacggcaatactcttttaccttcaacactcagtactatcactgcggcaaaagccattaatagtgatatagatatacttatgttaggatatggtatagaaagtattgcagttaaagccagtcatattcaggggatatccactgtatttgttgctgacagcccactattcgaacaccttctggcagagaatgtggaaaaacagatcagctattttttgaatagtgggaaaaatcattatcagtccattctttttcctgcaagctcttttggaaaaaactgtgcgccacgcttagcagcaaaacttgatgtatctcagattagtgatattacgcgtgtcattgatcaacatacctttgaacgtccaatctatgcgggtaacgctattgccacagtacacagtgatgatgaatataaagtgataactgttcgtcctacctcatttgcggctgcggttgcggaagatgagggaaaagcccccattgaatttacagaggttgttcttggttcagatcaggtaaaatttattagccaacatgttaataaaagtaaccgtccagacttacaatccgccagagttgtagtatcaggtggacgaggagtaggaagtgcacaggcattcaaagaacttgttgataacctggcagataaattaggcgctgccgtgggggcaagccgggctgctgtagatgctggttatgcaccaaatgattatcagattggccagaccggaaaaatcgttgcgcctcaactttatattgctctgggaatttctggcgccatacaacatcttgccgggatgaaagagtcaggcgtcattgtcgctattaataaggatcctgacgctcctatcttttctattgcggattatggtttggtagctgatattttcaaagctgtacctgaacttatcgataaattataa

SEQ ID NO: 18 - protein, 315 aa, Salmonella typhimurium
MASLVIAEHNGNTLLPSTLSTITAAKAINSDIDILMLGYGIESIAVKASHIQGISTVFVADSPLFEHLLAENVEKQISYFLNSGKNHYQSILFPASSFGKNCAPRLAAKLDVSQISDITRVIDQHTFERPIYAGNAIATVHSDDEYKVITVRPTSFAAAVAEDEGKAPIEFTEVVLGSDQVKFISQHVNKSNRPDLQSARVVVSGGRGVGSAQAFKELVDNLADKLGAAVGASRAAVDAGYAPNDYQIGQTGKIVAPQLYIALGISGAIQHLAGMKESGVIVAINKDPDAPIFSIADYGLVADIFKAVPELIDKL

SEQ ID NO: 19 - DNA, 1371 bp, Salmonella typhimurium
  CDS (1)..(1371)
atgaaggctaaatatttagcgctgatgatcggtgcttgcttttctcataacctttgggcagcgaataatatcactattgagcagcgtctggctgaactggagcaacgtgttgttaatgctgaaaaacgggcatccgatgccgaggcgcaaattcgctcgttgaaacagcagcaggtcgccgctacgccgacggtgaatgtccagtccgccgagcccattgcagcagataaaacgccgccgaagctgactttatccggattcagcgatattaagttctatggcgatgtcgaatttaatatggatgcggcaagccgttccggtagtctgacatcgacgagaacgtcagcgaataaagattgggcaccgggaaccaatgaacgctgggatattaacggacgcctgttgctgggctttgatggctaccagcggctggacaacggtaattttgccggattctctgtacagcctctgcggcacctgaccggaaaaatgaaccttgatgatgccgtttccttctttggtcgtgagaatgactggaaaattaaggttggtcgttttgaagcctacgatatgttcccgctgaatcaggatacgtttattgaatattcggggaatacagcgaacgatctttacagtgacggttacggctatatctatatgatgaaagaaggacggggacgtagcgacagaggcggtaacttcctgctgagtaaaaccatcgacaactggtatttcgaagttaacacattgctggaaaatggcagtacgttatataccgagaagcagtaccacggaatggatttaagcaacgataaaaacgtggcttacgtccgtccggttatcgcctggcaaaacgggcgtttttcaacggcgatagcgatggaaagtaacgtcgttaacaacgcctatggctattatgagaatgggaagtggatcgatcagtcagatcgtacgggctatggttttaccatgacctggaatggtcaaaaaactgacccggaagatggcgcagtgattaacctgaataccgcctatatggacgcgaccgatgagacagattttaccgccggggtgaatgcgttgtggcatcgatttgaactgggttatatctatgcgcataacaaaatcgaagcctttaatgctaccaatatcgatgccgtttgtgaggacgattgctgggtcaccgatcccggcaattatgacattcacactattcatgcctcatatttattccccaacgtgatggatatgaaaaactttaacatctacctcggtgcctatgcttcatgggtagaggccaatccgaataatggcgataacagtgaagatgcgcgttacggcgggcgtctgagattcaaatatttcttctga

SEQ ID NO: 20 - protein, 456 aa, Salmonella typhimurium
MKAKYLALMIGACFSHNLWAANNITIEQRLAELEQRVVNAEKRASDAEAQIRSLKQQQVAATPTVNVQSAEPIAADKTPPKLTLSGFSDIKFYGDVEFNMDAASRSGSLTSTRTSANKDWAPGTNERWDINGRLLLGFDGYQRLDNGNFAGFSVQPLRHLTGKMNLDDAVSFFGRENDWKIKVGRFEAYDMFPLNQDTFIEYSGNTANDLYSDGYGYIYMMKEGRGRSDRGGNFLLSKTIDNWYFEVNTLLENGSTLYTEKQYHGMDLSNDKNVAYVRPVIAWQNGRFSTAIAMESNVVNNAYGYYENGKWIDQSDRTGYGFTMTWNGQKTDPEDGAVINLNTAYMDATDETDFTAGVNALWHRFELGYIYAHNKIEAFNATNIDAVCEDDCWVTDPGNYDIHTIHASYLFPNVMDMKNFNIYLGAYASWVEANPNNGDNSEDARYGGRLRFKYFF

SEQ ID NO: 21 - DNA, 1620 bp, Salmonella typhimurium
  CDS (1)..(1620)
atgtttatacgggaaattacacgctggtttgccagacactcgtttcaaaaccggatatttctactgatcctgtttacatccaccatcgtgatgctggcaatcacctggtatctgaccgatatcacgaaagaacgcctgcattatcaggtgggccaacgtgcgcttattcaggcaatgcaaatatccgctatgccagaattagttgaagccgtagaagcgcatgatttgtcacgcatcaaagcgttgattgacccaatgcgctcgttttcggatgcgacttacattaccgttggcaacgaaaaaggtcagcgactgtatcacgtcaacccggatgagatcggcaaatatatggagggcggcgatagcgacgacgcgctgtataacgctaaaagctacgtatcggtgcgcaaaggttcattaggctcttccctacgtggtaaatccccgattcaggacagtaccggtaaagtcatcggtattgtttccgtcggctatactctggaacagttggagagctggcttaacttacaaatcagctcgttgctgttcccgatggcgttactgctgctggtactgttgtactgtgcccgccgcttctcgctgcatattaaaaagcagatgcttaacatggagccgcagcagctttcacaactattaattcagcaaagcgtgttatttgagtcggtatttgaagggttgatcgccattgactcccatcaccgtattaccgccattaaccaaacggctcgccgcttactgaatctgagccaaccggagtctgaactcattggctatgcaatccaccatgtcgtgttgccggaggccttcttttacgatgcgccgcaaaaaaacaaaaaagatgagatcgtcacctttaaccaaatgaaagtcatcgccagtcgcatggcggtcattattgacaaccagcctcagggctgggttattagctttcgcaacaaagacgatatcaatacactcagcctgcagttaagtcaggtgcggcaatacgccgataacctgcgcgccgtgcagcatgagcatcgcaaccttatttccaccattgccggactgctgttcctgaagcgctacgataatgcgctggcgctgattcagcagcagtcggaaagccaccagaaagtactcgattttatttcccataatttccgcgaccaccaccttgcggggctattaatcggtaaatattatcgggcaaaagagctggggctggagctgattttcgatccttcaagctttgttgaacacctgccgtcatcgctctcgcataatgaatggatttcgattgttggcaatttattggacaacgcctataacgccaccctgctcaatcgcgaaggctcgcgacagattgagtgcctgatcaacagcgaaggtaatgaagtcattattgaagttgccgatcagggatgtggtattgacgaaaatatccgccagcatattttcgagcgtggagtgaccacccacaacaacggcgatcacggaattggattatggttggtgcacagctacgtcaagcaggccaatggtgacattatcgtcgataacaataatccttttggcaccatttttaccctctatatcccttttaccagagaagaataa

SEQ ID NO: 22 - protein, 539 aa, Salmonella typhimurium
MFIREITRWFARHSFQNRIFLLILFTSTIVMLAITWYLTDITKERLHYQVGQRALIQAMQISAMPELVEAVEAHDLSRIKALIDPMRSFSDATYITVGNEKGQRLYHVNPDEIGKYMEGGDSDDALYNAKSYVSVRKGSLGSSLRGKSPIQDSTGKVIGIVSVGYTLEQLESWLNLQISSLLFPMALLLLVLLYCARRFSLHIKKQMLNMEPQQLSQLLIQQSVLFESVFEGLIAIDSHHRITAINQTARRLLNLSQPESELIGYAIHHVVLPEAFFYDAPQKNKKDEIVTFNQMKVIASRMAVIIDNQPQGWVISFRNKDDINTLSLQLSQVRQYADNLRAVQHEHRNLISTIAGLLFLKRYDNALALIQQQSESHQKVLDFISHNFRDHHLAGLLIGKYYRAKELGLELIFDPSSFVEHLPSSLSHNEWISIVGNLLDNAYNATLLNREGSRQIECLINSEGNEVIIEVADQGCGIDENIRQHIFERGVTTHNNGDHGIGLWLVHSYVKQANGDIIVDNNNPFGTIFTLYIPFTREE

SEQ ID NO: 23 - DNA, 819 bp, Salmonella typhimurium
  CDS (1)..(819)
atgagactcgcttcccgttttggtagacagaattccattcgtcgtgagagtcctttggctgacgctgaattaatgcagactgtgccttcagttttttccggagacaaacatgaatcccggagcgaacgttatacttatattccaaccattaatatcatcaacaggttacgtgaggaaggttttcagtcgttctttgcctgccagagtcgtgtacgtgatttaagtcgccgggaatacagtaaacatatgctgcgttttcgtcgtgaaggacagattaatgggaaagaggttccggaaattattttgctcaattctcatgacggttcgtcaagttatcagatggttcccgggctgttcagatttatctgtacaaatggactggtatgcggatcatttggtgaaatacgtgtgccacacaagggagatattgttgggcaggtgattgagggggattatgaggttatggggatcttcgataaagccaccgaaaatatggagtcaatgaagtcagtgatacttaatcaggatgagcaatatctgtttggtaaagcggcactgactgtcagatatgaggacgaaaataaaatccctgtttctcctgaacaaataattactccacgtcgtcgggaagataaacaaaatgacctgtggacaacatatcagcgtgtacagaagaatatgatgaagagtgggttacccggcaggaatgcctccggaaaaaacacccggatcagggcagttaccggtattaatggtgatatccggttaaacaaggcgctgtggatgattgctgaacagtttcgtgaatgtaagtcataa

SEQ ID NO: 24 - protein, 272 aa, Salmonella typhimurium
MRLASRFGRQNSIRRESPLADAELMQTVPSVFSGDKHESRSERYTYIPTINIINRLREEGFQSFFACQSRVRDLSRREYSKHMLRFRREGQINGKEVPEIILLNSHDGSSSYQMVPGLFRFICTNGLVCGSFGEIRVPHKGDIVGQVIEGDYEVMGIFDKATENMESMKSVILNQDEQYLFGKAALTVRYEDENKIPVSPEQIITPRRREDKQNDLWTTYQRVQKNMMKSGLPGRNASGKNTRIRAVTGINGDIRLNKALWMIAEQFRECKS

SEQ ID NO: 25 - DNA, 564 bp, Salmonella typhimurium
  CDS (1)..(564)
atgttaaagcgcgtgttttacagcctgttggtcctggtaggcttgctgctgttgacggtgcttggcctcgatcggtggatgagctggaaaacagcgccctatatctatgacgagctacaggatctcccctaccgccaggttggcgtcgtgttagggacagccaaatactatcgcaagggtgtcattaatcagtactatcgttatcgtatccagggcgcgttaaacgcttacaatagcggtaaagtcaattacctgctgctcagcggcgacaatgccctgcaaagctataacgaaccgatgaccatgcgcaaagaccttatcgcggcgggcgtcgatcctgcggacattgtgcttgattacgctggcttccgtacgcttgactccattgtgcgtacccgcaaagtgtttgataccaacgactttattatcatcactcagcgcttccactgcgaacgcgccttgtttattgcgttgcatatgggcattcaggcgcagtgttaccgcggtaccttcacccaaaaatatgctgacagtgcgcctgcgcgaatttggcgcccgctttag

SEQ ID NO: 26 - protein, 187 aa, Salmonella typhimurium
MLKRVFYSLLVLVGLLLLTVLGLDRWMSWKTAPYIYDELQDLPYRQVGVVLGTAKYYRKGVINQYYRYRIQGALNAYNSGKVNYLLLSGDNALQSYNEPMTMRKDLIAAGVDPADIVLDYAGFRTLDSIVRTRKVFDTNDFIIITQRFHCERALFIALHMGIQAQCYRGTFTQKYADSAPARIWRPL

(19) United States
(12) Patent Application Publication
(10) Pub. No.: US 2003/0157121 A1
(43) Pub. Date: Aug. 21, 2003

(54) Virulence genes, proteins, and their use

(76) Inventor: Steven Neville Chatfield, Berkshire (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/182,504

(22) PCT Filed: Feb. 2, 2001
(86) PCT No.: PCT/GB01/00449

(30) Foreign Application Priority Data
Feb. 3, 2000 (GB) 0002552.8
Feb. 3, 2000 (GB) 0002554.4
Feb. 3, 2000 (GB) 0002555.1
Feb. 3, 2000 (GB) 0002556.9
Feb. 3, 2000 (GB) 0002564.3
Feb. 3, 2000 (GB) 0002563.5
Feb. 3, 2000 (GB) 0002562.7
Feb. 3, 2000 (GB) 0002561.9
Feb. 3, 2000 (GB) 0002560.1
Feb. 3, 2000 (GB) 0002559.3
Feb. 3, 2000 (GB) 0002558.5
Feb. 3, 2000 (GB) 0002557.7
Sep. 20, 2000 (GB) 0023060.7
Sep. 20, 2000 (GB) 0023059.9

(51) Int. Cl. A61K 39/02; C07K 14/255; C12N 1/21; C07H 21/04; C07K 16/12

(52) U.S. Cl. 424/190.1; 435/252.3; 530/350; 536/23.7; 530/388.4

(57) ABSTRACT

A series of genes from Salmonella typhimurium are shown to encode products which are implicated in virulence. The identification of these genes threfore allows attenuated microorganisms to be produced. Furthermore, the genes or their encoded products can be used in the manufacture of vaccines for therapeutic application.

### FIELD OF THE INVENTION

This invention relates to virulence genes and proteins, and their use. More particularly, it relates to genes and proteins/peptides obtained from Salmonella typhimurium, and their use in therapy and in screening for drugs.

### BACKGROUND OF THE INVENTION

Salmonella typhimurium is one of the major causes of food poisoning, resulting in gastro-enteritis. The source of most infections is ingestion of contaminated water or food, such as poultry, eggs and diary products. Salmonella typhimurium is also able to cause typhoid in mice and to colonise the alimentary tracts of poultry. While infection of adult poultry leads to limited excretion of Salmonella in the faeces, infection of newly hatched chicks, which have a relatively simple gut flora, results in rapid multiplication and extensive excretion. This can lead to a rapid spread of a Salmonella strain through a flock as the housing and the water and feeding systems become contaminated. There is also a risk of contamination of the carcasses at slaughter, with entry of Salmonella typhimurium into human food. It is therefore desirable to provide means for treating or preventing conditions caused by Salmonella species in animals and humans e.g. by immunisation.

### SUMMARY OF THE INVENTION

The present invention is based on the discovery of virulence genes in Salmonella typhimurium.

According to a first aspect of the invention, a peptide of the invention is encoded by an operon including any of the nucleotide sequences identified herein as SEQ ID NOS. 1, 4, 6, 8, 11, 13, 15, 17, 19, 21, 23 and 25 of S. typhimurium or a homologue thereof in a Gram-negative bacterium, or a functional fragment thereof, for therapeutic or diagnostic use.

The peptides may have many therapeutic uses for treating Salmonella infections, including use in vaccines for prophylactic application.

According to a second aspect, a polynucleotide encoding a peptide defined above, may also be useful for therapy or diagnosis.

According to a third aspect, the genes that encode the peptides may be utilised to prepare attenuated microorganisms. The attenuated microorganisms will usually have a mutation that disrupts the expression of one or more of the genes identified herein, to provide a strain that lacks virulence. These microorganisms will also have use in therapy and diagnosis.

According to a fourth aspect, the peptides, genes and attenuated microorganisms according to the invention may be used in the treatment or prevention of a condition associated with infection by Salmonella or Gram-negative bacteria.

### DESCRIPTION OF THE INVENTION

The present invention is based on the discovery of genes encoding peptides which are implicated in virulence. The peptides and genes of the invention are therefore useful for the preparation of therapeutic agents to treat infection. It should be understood that references to therapy also include preventative treatments, e.g. vaccination. Furthermore, while the products of the invention are intended primarily for treatment of infections in human patients, veterinary applications are also considered to be within the scope of the invention.

The present invention is described with reference to Salmonella typhimurium. However, all the Salmonella strains, and many other Gram-negative bacterial strains are likely to include related peptides or proteins having amino acid sequence identity or similarity to those identified herein. Organisms likely to contain the peptides include, but are not limited to the genera Enterobacter, Klebsiella, Shigella and Yersinia.

Preferably, the peptides that may be useful in the various aspects of the invention have greater than a 40% similarity with the peptides identified herein. More preferably, the peptides have greater than 60% sequence similarity. Most preferably, the peptides have greater than 80% sequence similarity, e.g. 95% similarity. With regard to the polynucleotide sequences identified herein, related polynucleotides that may be useful in the various aspects of the invention may have greater than 40% identity with the sequences identified herein. More preferably, the polynucleotide sequences have greater than 60% sequence identity. Most preferably, the polynucleotide sequences have greater than 80% sequence identity, e.g. 95% identity.

The terms “similarity” and “identity” are known in the art. The use of the term “identity” refers to a sequence comparison based on identical matches between correspondingly identical positions in the sequences being compared. The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity.

Levels of identity between gene sequences and levels of identity or similarity between amino acid sequences can be calculated using known methods. In relation to the present invention, publicly available computer based methods for determining identity and similarity include the BLASTP, BLASTN and FASTA (Atschul et al., J. Molec. Biol., 1990; 215:403-410), the BLASTX program available from NCBI, and the Gap program from Genetics Computer Group, Madison, Wis. The levels of similarity and identity provided herein, were obtained using the Gap program, with a Gap penalty of 12 and a Gap length penalty of 4 for determining the amino acid sequence comparisons, and a Gap penalty of 50 and a Gap length penalty of 3 for the polynucleotide sequence comparisons.

Having characterised a gene according to the invention, it is possible to use the gene sequence to search for related genes or peptides in other microorganisms. This may be carried out by searching in existing databases, e.g. EMBL or GenBank.

Peptides or proteins according to the invention may be purified and isolated by methods known in the art. In particular, having identified the gene sequence, it will be possible to use recombinant techniques to express the genes in a suitable host. Active fragments and related molecules can be identified and may be useful in therapy. For example, the peptides or their active fragments may be used as antigenic determinants in a vaccine, to elicit an immune response. They may also be used in the preparation of antibodies, for passive immunisation, or diagnostic applications. Suitable antibodies include monoclonal antibodies, or fragments thereof, including single chain Fv fragments. Methods for the preparation of antibodies will be apparent to those skilled in the art.

Active fragments of the peptides are those that retain the biological function of the peptide. For example, when used to elicit an immune response, the fragment will be of sufficient size, such that antibodies generated from the fragment will discriminate between that peptide and other peptides on the bacterial microorganism. Typically, the fragment will be at least 30 nucleotides (10 amino acids) in size, preferably 60 nucleotides (20 amino acids) and most preferably greater than 90 nucleotides (30 amino acids) in size.

It should also be understood, that in addition to related molecules from other microorganisms, the invention encompasses modifications made to the peptides and polynucleotides identified herein which do not significantly alter the biological function. It will be apparent to the skilled person that the degeneracy of the genetic code can result in polynucleotides with minor base changes from those specified herein, but which nevertheless encode the same peptides. Complementary polynucleotides are also within the invention. Conservative replacements at the amino acid level are also envisaged, i.e. different acidic or basic amino acids may be substituted without substantial loss of function.

The preparation of vaccines based on attenuated microorganisms is known to those skilled in the art. Vaccine compositions can be formulated with suitable carriers or adjuvants, e.g. alum, as necessary or desired, to provide effective immunisation against infection. The preparation of vaccine formulations will be apparent to the skilled person. The attenuated microorganisms may be prepared with a mutation that disrupts the expression of any of the genes identified herein. The skilled person will be aware of methods for disrupting expression of particular genes. Techniques that may be used include insertional inactivation or gene deletion techniques. Attenuated microorganisms according to the invention may also comprise additional mutations in other genes, for example in a second gene identified herein or in a separate gene required for growth of the microorganism, e.g. an aro mutation or, with regard to Salmonella, in a gene located in the SPI2 region identified in WO-A-96/17951.

In a preferred embodiment, a double mutant comprises a mutation that disrupts the expression of either etfL (SEQ ID NO. 17) or sfiX (SEQ ID NO. 25), and a second mutation that disrupts a further virulence gene, e.g. a gene located within SPI-2. In a further preferred embodiment, the SPI2 gene is ssaJ. In an alternative embodiment, the second mutation disrupts the expression of an aro gene, e.g. aroA or aroC. In a further preferred embodiment, a double mutant comprises an attenuating mutations that disrupt the expression of etfL and sfiX.

Attenuated microorganisms may also be used as carrier systems for the delivery of heterologous antigens, therapeutic proteins or nucleic acids (DNA or RNA). In this embodiment, the attenuated microorganisms are used to deliver a heterologous antigen, protein or nucleic acid to a particular site in vivo. Introduction of a heterologous antigen, peptide or nucleic acid into an attenuated microorganism can be carried out by conventional techniques, including the use of recombinant constructs, e.g. vectors, which comprise polynucleotides that express the heterologous antigen or therapeutic protein, and also include suitable promoter sequences. Alternatively, the gene that encodes the heterologous antigen or protein may be incorporated into the genome of the organism and the endogenous promoters used to control expression.

More generally, and as is well known to those skilled in the art, a suitable amount of an active component of the invention can be selected, for therapeutic use, as can suitable carriers or excipients, and routes of administration. These factors would be chosen or determined according to known criteria such as the nature/severity of the condition to be treated, the type and/or health of the subject etc.

In a separate embodiment, the products of the invention may be used in screening assays for the identification of potential antimicrobial drugs or for the detection for virulence. Routine screening assays are known to those skilled in the art, and can be adapted using the products of the invention in the appropriate way. For example, the products of the invention may be used as the target for a potential drug, with the ability of the drug to inactivate or bind to the target indicating its potential antimicrobial activity.

The various products of the invention may also be used in veterinary applications, e.g. for the immunisation of poultry.

The following is a brief overview of the experimental procedure used to identify the virulence genes.

Mutants were generated via miniTn5 transposon insertion mutagenesis, as described in Hensel et al., Science, 1995; 269 (5222): 400-3. The S. typhimurium strain 12023 was mutated by conjugation with E. coli transformants containing signature-tagged mini-Tn5 plasmids. Mutant Salmonella were isolated by selection for resistance to kanamycin. These mutants were then screened for attenuated mutants in a mouse model of infection. Salmonella mutants containing mini-Tn5 insertions within the identified genes were not recovered from mice inoculated with a mixed population of mutants and are therefore likely to be attenuated.

The DNA regions flanking either side of the miniTn5 insertions were cloned either by inverse PCR, kanamycin-resistance marker rescue or random PCR. The DNA sequences obtained by subsequent DNA sequencing were compared against Salmonella typhimurium LT2 sequences in publicly available databases to help characterise the putative gene products.

The databases used were the NCBI database (http://www.ncbi.nlm.nih.gov/Microb_blast/unfunishedgenom e.html) and the Washington University School of Medicine database (http://genome.wustl.edu/gsc/Blast/client.pl).

The attenuation in virulence of mutants identified through the STM screen for mice was confirmed by subsequent infection experiments in mice and chickens. For mice infection studies, groups of BalbC mice were inoculated orally with 10⁸ cfu of the salmonella mutants and their survival was compared to mice infected with the wild-type 12023 strain. For attenuation of virulence for chickens, 10⁸ cfu of the mutants were inoculated orally into day-old hatchlings, and the percentage of birds surviving was compared to chicks inoculated with wild-type controls. Older chickens are more resistant to Salmonella infection and oral inoculation with strain 12023 results in colonisation but not mortality. Some of the mutants identified by the STM screen were also attenuated in colonisation. Groups of 20 three-week-old chicks were inoculated orally with 10⁸ cfu. The percentage of birds from which the mutant Salmonella were re-isolated, for up to 6 weeks after inoculation, was compared to chicks inoculated with the wild-type strain.

The following Examples illustrate the invention.

### EXAMPLE 1

A first mutant was identified and the nucleotide sequence immediately following the mini-Tn5 insertion was cloned and sequenced. The nucleotide sequence was used in a search of the Salmonella sequence database at Washington University and the NCBI database, and a gene sequence with 98% identity was obtained.

The gene sequence is shown as SEQ ID NO. 1 and two open reading frames were identified. The translated regions from each ORF is shown as SEQ ID NOS. 2 and 3.

The cloned nucleotide sequence was also used to search other databases and shows 81.2% identity to the yehS gene of E. coli K12 (EMBL accession number U00007), at nucleotides 24633-25014 of the latter.

This demonstrates that the disrupted region is at least partially identical to the yehS gene of E. coli. If Salmonella typhimurium has the same genetic organisation in this region as E. coli, then the methionine at position 17 of the 119 amino acid sequence represents the start codon of the yehS gene in Salmonella typhimurium. This means that the transposon has integrated 47 nucleotides upstream of the yehS coding sequence, which is exactly where the stop codon of the upstream gene yehT is located in E. coli (EMBL accession number U00007). The attenuation of the mutant could therefore be due to a polar effect of the transposon on yehS or disruption of a putative Salmonella yehT homologue.

### EXAMPLE 2

A further mutant was identified and the nucleotide sequence immediately following the mini-Tn5 insertion was cloned and sequenced.

The nucleotide sequence was used to search the Washington University and NCBI databases and a gene sequence having 99% identity was found. The gene sequence is shown as SEQ ID NO. 4. A translation of this region is shown as SEQ ID NO. 5.

The mutant was tested for attenuation of virulence. Survival of mice infected with the mutant strain was increased compared to that of mice infected with the wild-type.

### EXAMPLE 3

A further mutant was identified and the nucleotide sequence immediately following the mini-Tn5 insertion was cloned and sequenced.

The nucleotide sequence was used to search the Washington University and NCBI databases. A gene sequence having 98% identity was obtained. The gene sequence is shown as SEQ ID NO. 6. A translation of the sequence is shown as SEQ ID NO. 7.

### EXAMPLE 4

A further mutant was identified and the nucleotide sequence immediately following the mini-Tn5 insertion was cloned and sequenced.

The nucleotide sequence was used to search the Washington University and NCBI databases. A gene sequence having 99% identity was obtained. The gene sequence is shown as SEQ ID NO. 8. Two open reading frames were identified, and translations of each of these is shown as SEQ ID NOS. 9 and 10.

The cloned nucleotide sequence also shows 78.1% identity from nucleotide 23-219 to the yhjR gene of E. coli K12 (EMBL accession number U00039), at nucleotides 110460-110265 of the latter.

This demonstrates that the disrupted region is at least partially identical to the yhjR gene of E. coli. The transposon may be exerting a polar effect on yhjR.

The virulence gene was also tested for attenuation of virulence in one day old chickens. The percentage of chickens that survived oral infection with the mutant strain was increased to 55%, compared to only 20% of chickens surviving after infection with the wild-type strain.

### EXAMPLE 5

A further mutant was identified and the nucleotide sequence immediately following the mini-Tn5 insertion was cloned and sequenced.

The nucleotide sequence was used to search the Washington University and NCBI databases. A gene sequence having 97% identity was obtained, and is shown as SEQ ID NO. 11. A translation of this sequence is shown as SEQ ID NO. 12.

The cloned nucleotide sequence also shows 67.6% identity from nucleotide 282-389 to the asta gene of Campylobacter jejuni (EMBL accession number U38280) at nucleotides 1524-1632 of the latter.

The amino acid sequence shows 46.8% identity from amino acid 4-65 to AstA from Campylobacter jejuni (TrEMBL accession number Q46098) to amino acid 130-193 of the latter.

This demonstrates that the disrupted gene is at least partially identical to the asta gene from Campylobacter jejuni. The product of the asta gene is an arylsulfatase (Ruijin et al., J. Bacteriol., 1996; 178(11): 3335-8).

### EXAMPLE 6

A further mutant was identified and the nucleotide sequence immediately following the mini-Tn5 insertion was cloned and sequenced.

The nucleotide sequence was used to search the Washington University and NCBI databases. A gene sequence having 100% identity was obtained and is shown as SEQ ID NO. 13. A translation of this sequence is shown as SEQ ID NO. 14.

The cloned nucleotide sequence also shows 83.3% identity from nucleotide 1-36 to the nanA gene of E. coli K12 (EMBL accession number X03345) at nucleotides 540-575 of the latter.

This demonstrates that the disrupted gene is at least partially identical to the nanA gene from E. coli.

The NanA protein is a N-acetylneuraminate Lyase which converts sialic acid into pyruvate and N-acetyl mannosamine.

The mutant was tested for attenuation of virulence. Survival of mice infected with the mutant strain was increased compared to that of mice infected with the wild-type.

### EXAMPLE 7

A further mutant was identified and the nucleotide sequence immediately following the mini-Tn5 insertion was cloned and sequenced.

The nucleotide sequence was used to search the Washington University and NCBI databases. A gene sequence was identified having 100% identity, and is shown as SEQ ID NO. 15. An open reading frame were identified and the translation of this is shown as SEQ ID NO. 16.

The cloned nucleotide sequence also shows 74.7% identity from nucleotide 87-256 to the cinA gene of E. coli K12 (EMBL accession number D90856), at nucleotides 6006-5837 of the latter.

The transposon is inserted immediately upstream of the cloned nucleotide sequence. The phenotype of the mutant could be due to interruption of the gene or a polar effect on cinA.

The CinA protein is a putative competence damage protein (Martin et al., Mol. Microbiol., 1995; 15(2):367-79.

The mutant was tested for attenuation of virulence. Survival of mice infected with the mutant strain was increased compared to that of mice infected with the wild-type.

### EXAMPLE 8

A further mutant was identified and the transposon shown to be inserted within a gene shown to be 93% identical with the sequence identified as SEQ ID NO. 17. The translation of this sequence is shown as SEQ ID NO. 18.

The cloned nucleotide sequence also shows 58% identity from nucleotide 583-1208 to the etfL gene of Bradyrhizobium japonicum (EMBL accession number U32230), at nucleotides 1121-1740 of the latter.

This demonstrates that the disrupted gene is at least partially identical to the etfL gene of Bradyrhizobium japonicum.

The etfL gene of Bradyrhizobium japonicum codes for the large or alpha subunit EtfL (also called EtfA) of an electron transfer flavoprotein (SwissProt accession number P53573). The small or beta subunit of the protein is encoded by etfS (EMBL accession number U32230). Electron transfer flavoproteins (ETFs) are alpha-beta heterodimers found in eukaryotic mitochondria and bacteria (Tsai and Saier, Res. Microbiol., 1995; 146(5): 397-404).

The mutant was tested for attenuation of virulence. Survival of mice infected with the mutant strain was significantly increased compared to that of mice infected with the wild-type.

The mutant was also tested for attenuation of virulence in one-day old chickens. The percentage of chicks that survived oral infection with the mutant strain was increased to 85%, compared to only 20% of chicks surviving after infection with the wild-type strain. The mutant was also tested for the reduction of colonisation of older chickens. Five weeks after oral inoculation into 3 week-old chicks the percentage of birds that excreted the mutant strain was reduced to 20%, whereas 65% of birds inoculated with the wild-type strain at this time point excreted Salmonella.

### EXAMPLE 9

A further mutant was identified and the nucleotide sequence following the mini-Tn5 insertion was cloned and sequenced.

The nucleotide sequence was shown to be 99% identical to the sequence shown as SEQ ID NO. 19, identified from the Washington University and NCBI databases. A translation of the identified open reading frame is shown as SEQ ID NO. 20.

The cloned nucleotide sequence also shows 65.9% identity from nucleotide 210-401 to the rafY gene of E. coli (EMBL accession number U82290) at nucleotides 588-769 of the latter.

This demonstrates that the disrupted gene is at least partially identical to the rafY gene of E. coli.

The rafY gene of E. coli strain PS9 is located on the plasmid pRSD2, which enables E. coli to grow on raffinose. RafY (TrEMBL accession number P77076) is a general diffusion pore, that allows the diffusion of high molecular mass carbohydrates through the outer membrane (Andersen et al., Eur. J. Biochem., 1998; 254: 679-84).

The virulence gene was also tested for attenuation of virulence in one day old chickens. The percentage of chickens that survived oral infection with the mutant strain was increased to 45%, compared to only 20% of chickens surviving after infection with the wild-type strain.

### EXAMPLE 10

A further mutant was identified and the nucleotide sequence immediately following the mini-Tn5 insertion was cloned and sequenced.

The cloned nucleotide sequence was shown to be 99% identical to the gene shown as SEQ ID NO. 21. A translation of the open reading frame is shown as SEQ ID NO. 22.

The cloned nucleotide sequence also shows 74.2% identity from nucleotide 16-500 to the citA gene of Klebsiella pneumoniae (EMBL accession number U31464) at nucleotides 275-756 of the latter.

This demonstrates that the disrupted gene is at least partially identical to the citA gene from Klebsiella pneumoniae.

The mutant was tested for attenuation of virulence. Survival of mice infected with the mutant strain was increased compared to that of mice infected with the wild-type.

The gene was also tested for the reduction of colonisation of older chickens. Five weeks after oral inoculation into 3 week old chickens, the percentage of birds that excrete the mutant strain was reduced to 35%, whereas Salmonella were isolated from 65% of birds infected with the wild-type strain at this time point.

The citA gene of Klebsiella pneunomiae codes for a sensor kinase, capable of autophosphorylation in the presence of citrate and subsequently of phosphorylation of the response regulator CitB. CitA is essential for the expression of the citrate fermentation genes (Bott et al. Mol. Microbiol., 1995; 18: 533-46). A gene called citA has been cloned from Salmonella typhimurium (EMBL accession number D90203), but this gene encodes a citrate carrier (Shimamoto et al., J. Biochem., 1991; 110: 22-8).

### EXAMPLE 11

A further mutant was identified and the nucleotide sequence immediately following the mini-Tn5 insertion was cloned and sequenced.

The cloned nucleotide sequence was shown to be 100% identical to a gene identified as SEQ ID NO. 23. A translation of the open reading frame is shown as SEQ ID NO. 24.

The nucleotide sequence also shows 72.4% identity from nucleotide 34-218 to the gene32 of E. coli K12 (EMBL accession number X15279) at nucleotides 762-946 of the latter.

This demonstrates that the disrupted gene is at least partially identical to the gene32 of E. coli K12.

The amino acid sequence also shows 72.7% identity to the product of gene32 located on the F-plasmid of E. coli (SwissProt accession number P52149). Additionally it shows homology to two hypothetical proteins from E. coli K12, referred to as YfjQ and YafZ (SwissProt accession numbers P77206 and P52132).

A blast search against a database for unfinished genomes (Institute for Genomic Research TIGR) with the E. coli gene32 (EMBL accession number X15279) revealed the existence of a homologous sequence in the Salmonella typhimurium genome (gnl|WUGSC_{—}99287|stmlt2-.Contig1522), which shows 77.7% identity to the sequence from E. coli and 100% identity from nucleotide 37243-37426 to nucleotide 1-184 of the cloned 186 nucleotide sequence. A homologue of the E. coli gene32 also exists in Klebsiella pneumoniae (gnl|WUGSC_{—}573|kpneumo_B_KPN.Contig1705). The cloned 186 nucleotide sequence shows 64.7% identity from nucleotide 1-184 to nucleotide 3915-4099 of the latter.

The mutant was tested for attenuation of virulence. Survival of mice infected with the mutant strain was increased to 70% compared to only 20% of mice infected with the wild-type strain.

### EXAMPLE 12

A further mutant was identified and the transposon shown to be inserted within a gene shown to be 98% identical with the sequence identified herein as SEQ ID NO. 25. A translation of this sequence is shown as SEQ ID NO. 26.

The cloned nucleotide sequence also shows 98% identity from nucleotide 42-996 to the sfiX gene of Salmonella typhimurium strain LT2 (EMBL accession number AJ002276), at nucleotides 1-964 of the latter.

This demonstrates that the disrupted gene is at least partially identical to the sfiX gene of Salmonella typhimurium strain LT2.

Sequence identity was also shown with other homologues, including the SanA protein of E. coli (SwissProt accession number P33017).

It has been proposed that SfiX acts as part of a barrier that prevents vancomycin access to its site of action (peptidoglucan) (Mouslim et al., Mol. Gen. Genet., 1998; 259: 46-53). The homologous protein SanA from E. coli is also required for vancomycin-resistance at high temperatures (Rida et al., J. Bacteriol, 1996; 178(1): 94-102).

The mutant was tested for attenuation of virulence. Survival of mice infected with the mutant strain was increased compared to that of mice infected with the wild-type.

The mutant was also tested for attenuation of virulence in one-day old chickens. The percentage of chicks that survived oral infection with the mutant strain was increased to 75%, compared to only 20% of chicks surviving after infection with the wild-type strain. The mutant was also tested for the reduction of colonisation of older chickens. Five weeks after oral inoculation into 3 week-old chicks the percentage of birds that excrete the mutant strain was reduced to 25%, whereas salmonella were isolated from 65% of birds inoculated with the wild-type strain at this time point.

1. A peptide encoded by an operon including any of the nucleotide sequences identified herein as SEQ ID NOS. 1, 4, 6, 8, 11, 13, 15, 17, 19, 21, 23 and 25, of S. typhimurium, or a related molecule having at least 40% sequence similarity or identity at the peptide or nucleotide level in a Gram-negative bacterium, or a functional fragment thereof, for therapeutic or diagnostic use.

2. A peptide according to claim 1, wherein the sequence similarity or identity is at least 60%.

3. A peptide according to claim 1 or claim 2, wherein the sequence similarity or identity is at least 90%.

4. A peptide according to claim 1, comprising the amino acid sequence identified herein as SEQ ID NOS. 2, 3, 5, 7, 9, 10, 12, 14, 16, 18, 20, 22, 24 and 26.

5. A polynucleotide encoding a peptide according to any preceding claim, for therapeutic or diagnostic use.

6. A host transformed to express a peptide according to any of claims 1 to 4.

7. An attenuated microorganism comprising a mutation that disrupts the expression of any of the nucleotide sequences defined in claim 1.

8. A microorganism according to claim 7, wherein the mutation is insertional inactivation or a gene deletion.

9. A microorganism according to claim 7 or claim 8, wherein the microorganism is Salmonella spp.

10. A microorganism according to any of claims 7 to 9, comprising a second mutation in a second nucleotide sequence.

11. A microorganism according to claim 10, wherein the second mutation disrupts the expression of the ssaJ gene.

12. A microorganism according to claim 10, wherein the second mutation disrupts the expression of an aro gene.

13. A microorganism according to any of claims 7 to 12, for therapeutic or diagnostic use.

14. A microorganism according to any of claims 7 to 13, comprising a heterologous antigen, therapeutic peptide or nucleic acid.

15. A vaccine comprising a peptide according to any of claims 1 to 4, or the means for its expression.

16. A vaccine comprising a microorganism according to any of claims 7 to 14.

17. An antibody raised against a peptide according to any of claims 1 to 4.

18. Use of a product according to any of claims 1 to 14, for the manufacture of a medicament for use in the treatment or prevention of a condition associated with infection by Salmonella or Gram-negative bacteria.

19. Use according to claim 18, wherein the condition is gastro-enteritis.

20. Use according to claim 18 or claim 19, for veterinary treatment.

21. Use of a product according to any of claims 1 to 14, in a screening assay for the identification of an antimicrobial drug.
